(19) United States
(12) Patent Application Publication
(10) Pub. No.: US 2015/0024428 A1
(43) Pub. Date: Jan. 22, 2015

(54) METHOD OF IDENTIFICATION OF SPORE-FORMING Bacillus spp. BY DIRECT In-situ ANALYSIS OF MALDI-TOF MASS SPECTROMETRY, AND ANALYSIS SYSTEM

(71) Applicant: AGENCY FOR DEFENSE DEVELOPMENT, DAEJEON (KR)

(72) Inventors: Youngsu JEONG, Daejeon (KR); Jonghee LEE, Daejeon (KR); Sunkyung CHOI, Sejong City (KR); Juhyun KIM, Seongnam-si (KR)

(73) Assignee: AGENCY FOR DEFENSE DEVELOPMENT, DAEJEON (KR)

(21) Appl. No.: 14/034,647

(22) Filed: Sep. 24, 2013

(30) Foreign Application Priority Data
Jul. 16, 2013 (KR) 10-2013-0083347

(51) Int. Cl. H01J 49/40; C12Q 1/04

(52) U.S. Cl. CPC H01J 49/40; C12Q 1/04
USPC 435/34

(57) ABSTRACT

A method of the identification of Bacillus species by direct in-situ analysis of MALDI-TOF MS (Matrix-Assisted Laser Desorption/Ionization Time-Of-Flight Mass Spectometry) in which spore-forming bacteria are applied intact without any pretreatment, and an analysis system of distinctive biomarkers which allow Bacillus spores to be distinguished. Rapid and accurate detection and identification of Bacillus species can be achieved by the method and analysis system.

### CROSS REFERENCE TO RELATED APPLICATION

This application claims the benefit of Korean Patent Application No. 10-2013-0083347, filed on Jul. 16, 2013, entitled “A method of identification of spores-forming Bacillus using in-situ MALDI-TOF mass spectrometer and analysis system”, which is hereby incorporated by reference in its entirety into this application.

### BACKGROUND Of THE INVENTION

1. Technical Field

The present invention relates to a method of the identification of spore-forming Bacillus spp. by direct in-situ analysis of MALDI-TOF MS (Matrix-Assisted Laser Desorption/Ionization Time-Of-Flight Mass Spectrometry) in which spore-forming bacteria are directly applied to MALDI-TOF without any pretreatment, and an analysis system therefor.

2. Description of the Related Art

Members of the genus Bacillus are rod-shaped bacteria with catalase activity. To cope with stressful environmental conditions, the cells produce endospores, showing facultative anaerobic properties. The genus Bacillus contains two important groups of bacteria named after Bacillus subtilis and Bacillus cereus.

Being one of the best understood prokaryotes in terms of molecular biology and cell biology, the clade of Bacillus subtilis is used as renowned model organisms for genetic research.

One clade, formed by Bacillus cereus, B. thuringiensis, B. antrophaeus, and B. amyloliquefaciens, under current classification standards, exhibits very high similarity in terms of phenotype and phylogeny so that they are very difficult to distinguish.

B. anthracis is a Gram-positive, endospore-forming bacterium. This microorganism acts as the etiologic agent of anthrax, a significant common disease of livestock and humans, and is a possible agent in biological warfare and bioterrorism.

Biochemical, chemotaxonomic, physiological, and genomic methods are typically used for the identification a microorganisms. Nevertheless, novel, accurate and rapid methods for the identification of bacteria are of great significance since conventional techniques cannot promise rapid, accurate classification and identification of bacteria.

MALDI-TOF (matrix-assisted laser desorption/ionization time-of-flight) mass spectroscopy (MALDI-TOF MS) is a technique configured to allow the rapid analysis of components on the basis of the time for which specimens after being ionized, reach the detector in the flight tube.

The MALDI Biotyper developed by Bruker, Germany can identify various colonies according to species at high speed by comparing the protein information obtained by MALDI-TOF mass spectrometry with preexisting data constructed for the colonies.

It takes as short as 6 min on average per strain for MALDI-TOF mass spectrometry to identify a microorganism, while the expense of identification by MALDI-TOF mass spectrometry accounts for 22˜32% of that required by the conventional methods including commercially available kits. Therefore, MALDI-TOF mass spectrometry is convenient for identifying microorganisms, in a short time with low expense.

Recent studies on microorganism identification using various MALDI-TOF mass spectrometric techniques have been directed toward direct whole cell mass spectrometry, in-situ analysis on vegetative cells or colonies without particular pretreatment. For microorganisms forming spores, however, the conventional methods cannot allow identification without the application of various pretreatments including extraction.

To solve this problem, one study suggested the use of a high-resolution MALDI-TOF mass spectrometer in identifying spore-forming bacteria. However, this is difficult to industrially apply because MALDI-TOF mass spectrometers are large in size, and highly expensive.

### PRIOR ART DOCUMENT

### Non-patent Documents

(Non-patent document 1) Barbuddhe, S. B.; Maier, T.; Schwarz, G.; Kostrzewa, M.; Hof, H., Domann, E.; Chakraborty, T.; Hain, T. Appl. Environ. Microbiol. 2008, 74, 5402-5407.

(Non-patent document 2) Fenselau, C., Demirev, P. A. Mass Spectrom. Rev. 2001, 20, 157-171.

(Non-patent document 3) He, Y.; Chang, T. C.; Li, H.; Shi, G.; Tang, Y.-W. Can. J. Microbiol. 2011, 57, 533-538.

(Non-patent document 4) Moura, H.; Woolfitt, A. R.; Carvalho, M. G.; Pavlopoulos, A.; Teixeira, L. M.; Satten, G. A.; Barr, J. R. FEMS Immunol. Med. Microbiol. 2008, 53, 333-342.

(Non-patent document 5) Lasch, P.; Beyer, W.; Nattermann, H.; Stammler, M.; Siegbrecht, E.; Grunow, R.; Naumann, D. Appl. Environ. Microbiol. 2009, 75, 7229-7242.

(Non-patent document 6) Aemirev, P. A.; Feldman, A. B.; Kowalski, P.; Lin, J. S. Anal. Chem. 2005, 77, 7455-7461.

### SUMMARY OF THE INVENTION

It is an object of the present invention to provide a method of rapidly and easily identifying spore-forming Bacillus bacteria using a matrix-assisted laser desorption/ionization time-of-flight mass spectrometer (hereinafter referred to as “MALDI-TOF MS”).

It is another object of the present invention to provide an analysis system of Bacillus spores.

In accordance with an aspect thereof, the present invention provides a method of identifying a Bacillus species using a matrix-assisted laser desorption/ionization time-of-flight mass spectrometer (MALDI-TOF MS) comprising: directly spotting a sample containing an intact spore-forming Bacillus bacterium onto a MALDI target plate without conducting any pretreatment thereto; and performing MALDI-TOF mass spectroscopy to acquire spectral data of the sample; and analyzing the spectral data with reference to a reference data (m/z) to identify the sample, said reference data being established for biomarker peaks distinctive for known Bacillus species.

In accordance with another aspect thereof, the present invention provides an analysis system for specific Bacillus spores, comprising mass spectrum peak data in terms of mass-to-charge ratio.

Employing spore-forming Bacillus bacteria themselves as samples without pretreatment given thereto, the method and analysis system of the present invention can detect and identify specific Bacillus bacteria rapidly and accurately.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a schematic diagram of the direct in-situ analysis of mass spectra of Bacillus species spores.

FIG. 2 shows mass spectra of Bacillus spores prepared using different sample preparation methods as described in the Example section.

FIG. 3 shows mass spectra of five different Bacillus spores.

### DESCRIPTION OF THE PREFERRED EMBODIMENTS

A detailed description is given of the present invention, below.

In accordance with an aspect thereof, the present invention addresses a method of identifying Bacillus bacteria, using MALDI-TOF MS, wherein the a sample including a spore-forming Bacillus bacterium is directly spotted onto a MALDI target plate without any pretreatment given thereto.

In the method, a matrix solution may be applied to the target plate and dried after the sample is spotted.

The MALDI-TOF MS useful in the present invention may preferably be Autoflex Speed LRF mass spectrometer, manufactured by Bruker, but is not limited thereto.

The instrument may be equipped with a 355 nm Nd/YAG laser operating at 337 nm at pulse rates of up 1 kHz.

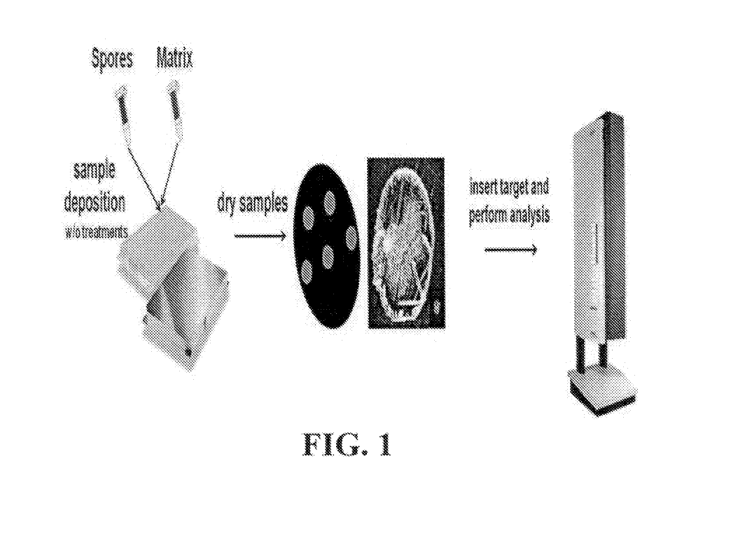

Application of the MALDI-TOF MS is schematically depicted in FIG. 1.

As used herein, the term “MALDI target plate” refers to one of MALDI-TOF MS parts onto which an analyte and a matrix solution for helping ionize the analyte are spotted, and can be readily understood to a person having ordinary knowledge in the art. For details, reference may be made to the website of Brucker (http://www.bruker.com/search.html).

Examples of the matrix useful in the present invention include DHB (dihydroxybenzoic acid), sinapinic acid, THAP (trihydroxy acetophenone), HABA (hydroxyphenylazo benzoic acid), dithranol, CHCA (cyano-hydroxycinnamic acid), RA (all-trans-retinoic acid), and IAA (indoleacrylic acid), but are not limited thereto.

Given spore-forming bacteria rather than general microorganisms, conventional MALDI-TOF mass spectroscopy needs a more complicated procedure for cell disruption and extraction due to the hard structure of spores, thus consuming greater time and requiring additional labor for sample pretreatment, both of which are disadvantageous.

As opposed to the conventional MALDI-TOF mass spectrometry, the present invention is characterized by directly in-situ spotting Bacillus spore samples onto a MALDI target plate without a pretreatment in identifying Bacillus bacteria.

After the spotting, the MALDI target plate with the dried sample applied to a MALDI-TOF mass spectrometer to identify Bacillus bacteria.

The method of identifying Bacillus bacteria in accordance with the present invention employs a laser power higher than that used in the analysis of general microorganisms, for example, E. coli.

Compared to that for the analysis of E. coli, the laser power necessary for analyzing Bacillus bacteria is at least 1.6-fold higher, and preferably 2.5- to 2.7-fold higher. When the laser power hi lower than 1.6 times that used for E. coli, it is impossible to perform direct in-situ mass analysis due to the hardness of spores. On the other hand, a laser power higher than 2.7 times that used for E. coli increases noise upon acquisition of the spectrum.

The MBT_FC parameter of the Autoflex Speed LRF MALDI-TOF mass spectrometer is preferably set to have a laser power of as large as or more than 50%, and more preferably been 70 to 80%. When a Bacillus sample is pretreated as in conventional methods, the laser power in the MBT-FC parameter may be decreased to 30%.

With the laser power lower than 50% in the MBT_FC parameter, the direct in-situ mass analysis cannot be performed since hard spores are not sufficiently broken down. On the other hand, when the laser power is greater than 80%, spectral data is acquired with increased noise.

Within the scope of the Bacillus bacteria useful in the present invention are Bacillus anthracis, Bacillus cereus, Bacillus globigii, Bacillus subtilis and Bacillus thuringiensis. However, as long as it produces an endospore, any Bacillus bacterium may be used as an analyte.

Employing spore-forming Bacillus bacteria themselves as samples, the method of the present invention can identify specific Bacillus bacteria by comparing mass-to-charge ratios (m/z) of the samples with those of the biomarkers of Bacillus bacteria.

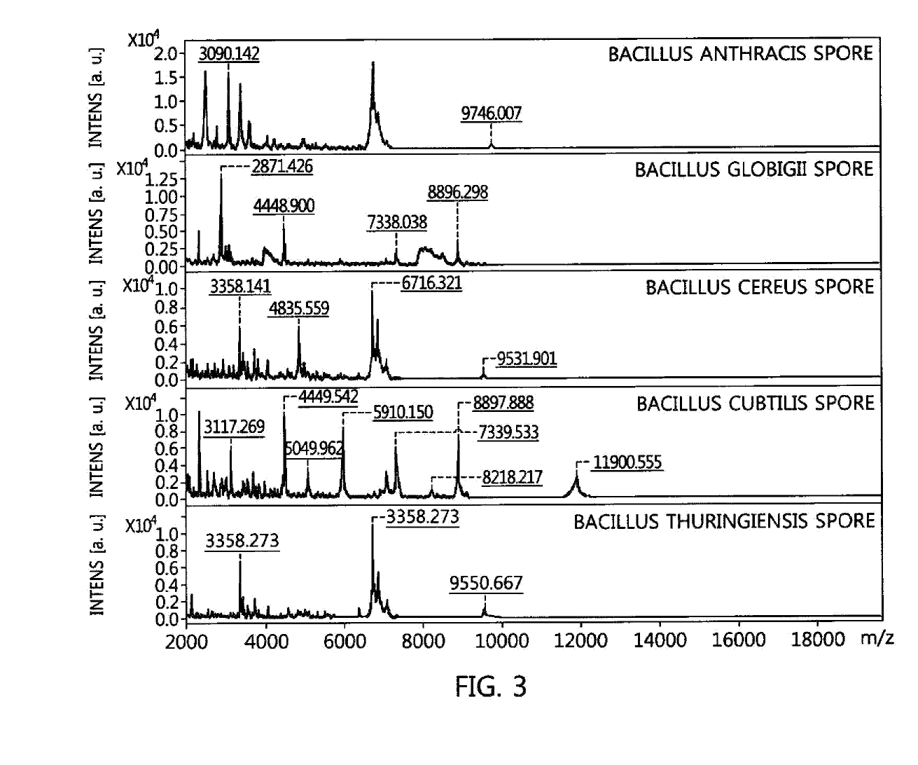

In one embodiment of the present invention, biomarkers which are reproducibly detected with significance can be identified according to Bacillus spore (FIG. 3 and Table 1).

That is, the five kinds of Bacillus spores exhibit distinctive mass spectrum patterns in the mass range of from 2,000 to 20,000 Da, with distinct peaks of biomarkers specific therefor (FIG. 3). Thus, the present invention envisages an analysis system based on these distinct peaks of biomarkers.

Mass-to-charge ratios (m/z) of the biomarkers and standard errors thereof, and relative detection intensity at each biomarker and standard errors thereof are given in Table 1 where important biomarkers with high detection intensity are marked red.

Interestingly, B. globigii spores were found to have a mass range of from 7.9 kDa to 8.5 kDa, with a considerably broad and distinctive mass pattern, which is expected to be very helpful in identifying B. globigii spores. The distinct spectral pattern of B. globigii is marked by a yellow block in Table 1.

Also, Bacillus anthracis spores, possibly used as a biological weapon, was clearly distinguishable from those of Bacillus cereus and Bacillus thuringiensis, both very close in phylogeny thereto.

In greater detail, the spectrum for Bacillus anthracis spores yielded main mass peaks useful as distinctive biomarkers thereof preferably at mass-to-charge ratios (m/z) of 2196, 2473, 2503, 2786, 3089, 3376, 3594, 6684, 6753, 6840, and 9746, and more preferably at mass-to-charge ratios (m/z) of 2080, 2097, 2196, 2446, 2473, 2503, 2518, 2523, 2579, 2786, 3075, 3089, 3150, 3341, 3376, 3576, 3594, 3653, 4031, 4196, 4328, 4383, 4554, 4956, 5263, 5541, 6684, 6699, 6753, 6840, and 9746.

For Bacillus cereus sores, distinctive mass peaks preferably detected at mass-to-charge ratios (m/z) of 3357 3419, 3709, 4836, 4953, 6714, 6839, and 7085, and more preferably at mass-to-charge ratios (m/z) of 2109, 2123, 3079, 3193, 3357, 3419, 3542, 3709, 3807, 4031, 4335, 4425, 4836, 4953, 5173, 6714, 6839, and 7085 are given as biomarkers.

For Bacillus globigii spores, distinctive mass peaks preferably detected at mass-to-charge ratios (m/z) of 2324, 2870, 2886, 2992, 3123, 4447, 7907, 8053, 8199, 8345, 8492, 8895, and more preferably at mass-to-charge ratios (m/z) of 2324, 2870, 2886, 2918, 2934, 2992, 3123, 3430, 3760, 4418, 4447, 4682, 5047, 7072, 7336, 7907, 8053, 8199, 8345, 8492, and 8895 are detected as distinct biomarkers.

For Bacillus subtilis spores, distinctive mass peaks preferably detected at mass-to-charge ratios (m/z) of 2324, 2720, 2871, 2887, 2935, 3116, 5299, 5948 7338, 8201, 8347, 8494, 8896, and 11898, and more preferably at mass-to-charge ratios (m/z) of 2324, 2720, 2871, 2887, 2919, 2935, 2991, 3116, 3578, 4448, 5048, 5299, 5948, 7073, 7338, 8201, 8347, 8494, 8896, 11056, and 11898 are detected as distinct biomarkers.

For Bacillus thuringiensis spores, distinctive mass peaks preferably detected at mass-to-charge ratios (m/z) of 2109, 2127, 3357, 3419, 3709, 6715, 6840 and 7086, and more preferably at mass-to-charge ratios (m/z) of 2109, 2127, 2266, 2380, 2529, 3095, 3357, 3419, 3542, 3709, 4031, 4335, 4425, 4837, 4971, 5173, 6352, 6715, 6840, and 7086 are detected as distinct biomarkers.

The method of the identification of Bacillus bacteria, and the analysis system of Bacillus spores in accordance with the present invention make sure of the convenient and accurate identification of specific Bacillus bacteria even with significantly reduced labor and time.

A better understanding of the present invention may be obtained through the following examples which are set forth to illustrate, but are not to be construed as limiting the present invention.

### EXAMPLE 1

### Culturing of Bacillus Bacteria

For use in the present invention, Bacillus anthracis, Bacillus cereus, Bacillus globigii, Bacillus subtilis and Bacillus thuringiensis were granted from the Korea Centers for Disease Control and Prevention. For sporulation, a single colony of each strain was inoculated into a nutrient broth sporulation medium, and cultured at 32° C. for 2˜4 days with agitation. Culturing was continued until the cells showed more than 99% spore formation, as measured by optical microscopy. The spores thus formed were collected by centrifugation for removal of remnant vegetative cells and cell debris. Sporulation and spore purification were committed under an optical microscope with 400 magnification. The spores were obtained with a purity of 80˜90%. They were suspended at a density of 1×10⁹ CFU/ml in distilled water, and stored at 4° C. until use in experiments.

### EXAMPLE 2

To phylogenetically classify the spores by MALDI-TOF mass spectrometry, 1 μl of B. anthracis spores prepared at a density of 1×10⁸⁻⁹ CFU/ml was directly spotted onto MTP 384 target ground steel TF (Bruker Daltonics, Germany) without any pretreatment procedure, and dried at room temperature for 5 min, as shown in FIG. 1. Subsequently, 1 μl of a matrix solution prepared by dissolving a matrix (α-cyano-4-hydroxycinnamic acid (CHCA)) at a concentration of 12 mg/ml in TA₂, a 2:1 (vol/vol) mixture of trifluoroacetic acid (TFA, Sigma USA) and acetonitrile (CAN, Sigma, USA) was applied to each dried spore spot on the MALDI target plate, and then allowed to dry at room temperature for 5 min.

Mass spectra of the spores were obtained using the Autoflex Speed LRF mass spectrometer from Bruker Germany. The pulse ion extraction time was 200 ns. Spectral measurements were carried out in the linear mode of the MBT_FC parameter using an acceleration voltage of 19.51 kV and 18.26 kV at ion sources 1 and 2, respectively. A laser power in the MBT_FC parameter was equipped to 77%.

Reliable mass spectra for spore analysis could not he obtained when the laser power in the MBT_FC parameter was below 50%.

The lens voltage was 7.00 kV. The mass spectra of spores were stored in the low mass range between 0.5 and 2 kDa and in the intermediate mass range between 2 and 20 kDa. Escherichia coli DH5a (Bruker Daltonics, Germany) was used as a reference strain for mass calibration, with a peak tolerance of about 1000 ppm. At least 200 laser shots were co-added for each spore spectrum. Mass spectra for each spore were processed by smoothing, baseline subtraction, and intensity normalization using Bruker's Flex Control software package (v. 3.3; Bruker Daltonics). The smoothing and baseline subtraction were done by using Savitzky Golay algorithm and TopHat algorithm, respectively. For comparative analysis of spore biomarkers, the intensity of each peak by mass-to-charge ratio was evaluated using the Centroid algorithm. In the biomarker analysis, mass spectrum data of each spore were obtained in more than 28 different runs of experiments to confirm the reproducibility of peak patterns.

### COMPARATIVE EXAMPLE 1

The sample preparation of Example 2 according to the present invention was compared with other sample preparations for MALDI-TOF MS. In this regard, the same procedure as in Example 2 was carried out with the exception that an inactivation method, or a modified method combined with bead beating and trifluoroacetate (TFA) extraction was used to prepare samples to he spotted onto the MALDI plate.

As the inactivation method, as modified TFA inactivation method was used as described in P. Lasch, et. al., 2009. In the combined method of bead beating and trifluoroacetate (TFA) extraction, first, 30 μl of absolute ethanol (Merck, Germany) was mixed with 5 μl of a spore sample by vortexing for 5 min, followed by centrifugation at 13,000 rpm for 3 min. After removal of the supernatant, the ethanol was allowed to vaporize at room temperature for 3 min to dry the pellet. The spores were mixed by vortexing for 5 min with a small amount of beads (7 μl) and acetonirile (ACN, 7 μl) to ensure effective purification by mechanical shear force. Again, the mixture was vortexed for 10 min, together with 7 μl of 70% formic acid (Sigma, USA). For comparison, each of the spore samples prepared using the three methods (direct in-situ mass analysis, inactivation, and extraction) was spotted onto the MALDI target plates in at least triplicate.

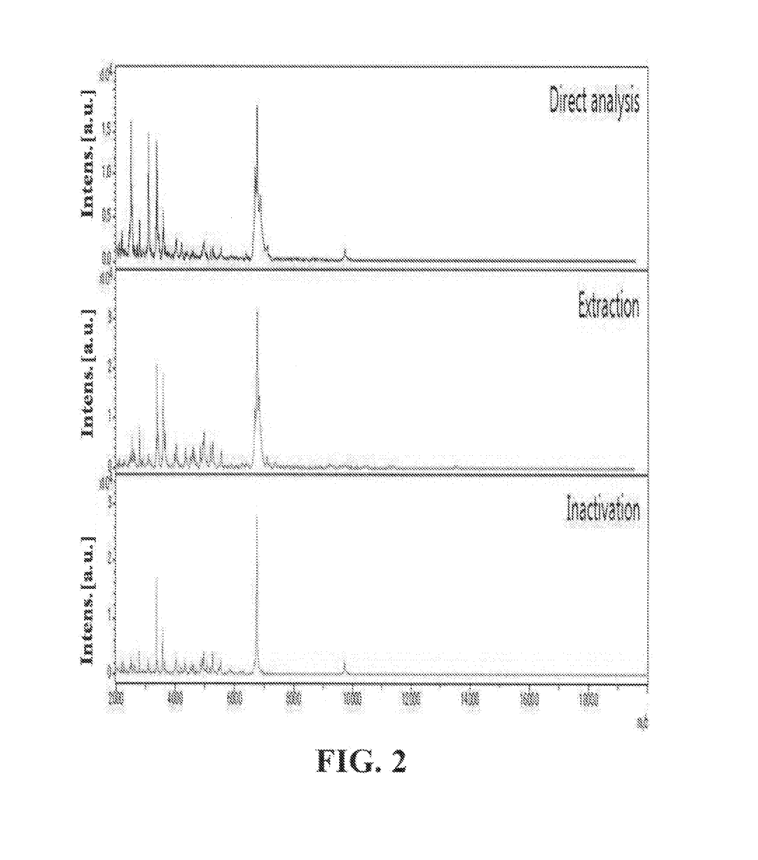

Results obtained in Example 2 and Comparative Example 1 are shown in FIG. 2. As is understood from the spectral data of FIG. 2, the method of the present invention characterized by directly spotting spore-forming Bacillus bacteria onto the MALDI target plate without pretreatment produced more abundant distinct peaks, compared to conventional methods requiring pretreatments.

### EXAMPLE 3

Mass peak profiles of the five different Bacillus spores, that is, Bacillus anthracis, Bacillus cereus, Bacillus globigii, Bacillus subtilis and Bacillus thuringiensis were obtained using the MALDI-TOF MS and analyzed in the same manner as in Example 2 to confirm the discrimination of them from one another.

The results are summarized in Table 1, below.

TABLE 1

| | |
|---|---|
| B. anthracis | B. cereus |

| | Bio | | Relative | | Bio | | Relative | |
|---|---|---|---|---|---|---|---|---|
| | markers | STDEV | intensity | STDEV | markers | STDEV | intensity | STDEV |
| m/z | 2080.66 | ±0.41 | 0.11 | ±0.05 | 2108.78 | ±0.49 | 0.19 | ±0.14 |
| | 2096.97 | ±0.47 | 0.11 | ±0.04 | 2123.44 | ±3.58 | 0.19 | ±0.16 |
| | 2196.16 | ±0.47 | 0.19 | ±0.10 | 3079.19 | ±0.66 | 0.04 | ±0.01 |
| | 2446.03 | ±0.70 | 0.12 | ±0.05 | 3103.42 | ±0.78 | 0.03 | ±0.01 |
| | 2473.07 | ±0.52 | 0.30 | ±0.08 | 3356.79 | ±0.56 | 0.24 | ±0.05 |
| | 2503.17 | ±0.52 | 0.65 | ±0.24 | 3418.83 | ±0.57 | 0.13 | ±0.03 |
| | 2517.88 | ±0.57 | 0.35 | ±0.12 | 3542.03 | ±0.66 | 0.10 | ±0.03 |
| | 2523.19 | ±0.66 | 0.33 | ±0.11 | 3708.67 | ±0.71 | 0.14 | ±0.05 |
| | 2579.21 | ±0.49 | 0.16 | ±0.05 | 3807.23 | ±0.72 | 0.09 | ±0.08 |
| | 2786.00 | ±0.54 | 0.32 | ±0.08 | 4031.21 | ±0.92 | 0.06 | ±0.01 |
| | 3075.22 | ±0.58 | 0.26 | ±0.06 | 4335.03 | ±0.71 | 0.09 | ±0.02 |
| | 3089.28 | ±0.58 | 0.44 | ±0.11 | 4424.50 | ±0.57 | 0.15 | ±0.04 |
| | 3150.47 | ±0.59 | 0.10 | ±0.03 | 4836.27 | ±0.62 | 0.15 | ±0.03 |
| | 3341.10 | ±0.64 | 0.22 | ±0.09 | 4953.16 | ±0.68 | 0.11 | ±0.03 |
| | 3375.69 | ±0.66 | 0.28 | ±0.08 | 5173.10 | ±0.70 | 0.07 | ±0.02 |
| | 3576.24 | ±0.72 | 0.26 | ±0.09 | 6714.36 | ±0.91 | 1.00 | ±0.00 |
| | 3593.73 | ±0.71 | 0.52 | ±0.17 | 6839.01 | ±1.07 | 0.70 | ±0.03 |
| | 3653.23 | ±0.79 | 0.12 | ±0.04 | 7085.05 | ±0.16 | 0.26 | ±0.04 |
| | 4030.99 | ±0.89 | 0.16 | ±0.05 | | | | |
| | 4195.93 | ±0.75 | 0.13 | ±0.02 | | | | |
| | 4327.88 | ±0.86 | 0.18 | ±0.08 | | | | |
| | 4382.51 | ±0.93 | 0.12 | ±0.03 | | | | |
| | 4553.50 | ±1.01 | 0.09 | ±0.02 | | | | |
| | 4956.00 | ±0.98 | 0.18 | ±0.03 | | | | |
| | 5263.00 | ±1.00 | 0.15 | ±0.03 | | | | |
| | 5540.56 | ±1.13 | 0.09 | ±0.01 | | | | |
| | 6683.73 | ±1.27 | 0.98 | ±0.04 | | | | |
| | 6698.86 | ±2.22 | 0.60 | ±0.06 | | | | |
| | 6753.46 | ±1.36 | 0.94 | ±0.07 | | | | |
| | 6839.77 | ±1.50 | 0.83 | ±0.06 | | | | |
| | 9745.74 | ±2.13 | 0.06 | ±0.01 | | | | |

| B. thuringiensis | | | | B. globigii | | | | B. subtilis | | | |
|---|---|---|---|---|---|---|---|---|---|---|---|
| Bio | | Relative | | Bio | | Relative | | Bio | | Relative | |
| markers | STDEV | intensity | STDEV | markers | STDEV | intensity | STDEV | markers | STDEV | intensity | STDEV |
| 2108.93 | ±0.65 | 0.12 | ±0.03 | 2324.17 | ±0.40 | 0.52 | ±0.26 | 2324.43 | ±0.53 | 0.88 | ±0.14 |
| 2126.59 | ±0.57 | 0.30 | ±0.19 | 2870.15 | ±0.55 | 0.88 | ±0.18 | 2720.02 | ±1.03 | 0.23 | ±0.10 |
| 2266.09 | ±0.59 | 0.07 | ±0.03 | 2886.32 | ±0.46 | 0.80 | ±0.16 | 2870.75 | ±0.79 | 0.73 | ±0.13 |
| 2379.60 | ±0.66 | 0.12 | ±0.08 | 2918.40 | ±0.45 | 0.26 | ±0.07 | 2886.92 | ±0.68 | 0.90 | ±0.14 |
| 2528.89 | ±0.59 | 0.22 | ±0.16 | 2934.46 | ±0.33 | 0.24 | ±0.07 | 2918.93 | ±0.72 | 0.30 | ±0.09 |
| 3095.14 | ±0.63 | 0.22 | ±0.17 | 2992.31 | ±0.77 | 0.25 | ±0.15 | 2935.14 | ±0.71 | 0.50 | ±0.11 |
| 3356.72 | ±0.64 | 0.31 | ±0.05 | 3122.69 | ±0.47 | 0.49 | ±0.27 | 2991.48 | ±0.61 | 0.26 | ±0.04 |
| 3418.82 | ±0.67 | 0.11 | ±0.03 | 3430.19 | ±0.61 | 0.16 | ±0.07 | 3116.29 | ±0.64 | 0.50 | ±0.10 |
| 3541.98 | ±0.67 | 0.08 | ±0.02 | 3759.76 | ±0.56 | 0.27 | ±0.09 | 3528.22 | ±0.74 | 0.21 | ±0.05 |
| 3708.64 | ±0.65 | 0.18 | ±0.07 | 4418.42 | ±0.56 | 0.23 | ±0.11 | 4447.75 | ±0.87 | 0.15 | ±0.06 |
| 4031.11 | ±0.70 | 0.06 | ±0.01 | 4447.29 | ±0.61 | 0.60 | ±0.32 | 5048.26 | ±0.88 | 0.14 | ±0.05 |
| 4334.98 | ±0.71 | 0.07 | ±0.02 | 4681.63 | ±0.75 | 0.14 | ±0.06 | 5299.15 | ±0.74 | 0.19 | ±0.06 |
| 4424.56 | ±0.67 | 0.09 | ±0.02 | 5047.38 | ±0.81 | 0.22 | ±0.09 | 5948.39 | ±1.04 | 0.26 | ±0.09 |
| 4837.39 | ±0.74 | 0.08 | ±0.02 | 7071.86 | ±0.96 | 0.16 | ±0.07 | 7073.19 | ±1.13 | 0.15 | ±0.06 |
| 4971.42 | ±0.75 | 0.08 | ±0.02 | 7336.33 | ±1.03 | 0.22 | ±0.10 | 7337.78 | ±1.16 | 0.27 | ±0.10 |
| 5172.84 | ±0.66 | 0.05 | ±0.01 | 7906.83 | ±1.32 | 0.20 | ±0.11 | 8201.02 | ±1.44 | 0.36 | ±0.11 |
| 6352.44 | ±0.74 | 0.05 | ±0.00 | 8053.02 | ±1.36 | 0.23 | ±0.13 | 8347.19 | ±1.52 | 0.23 | ±0.07 |
| 6714.57 | ±0.86 | 1.00 | ±0.00 | 8199.33 | ±1.28 | 0.20 | ±0.11 | 8493.71 | ±1.52 | 0.18 | ±0.07 |
| 6840.21 | ±1.08 | 0.54 | ±0.04 | 8345.36 | ±1.46 | 0.11 | ±0.06 | 8896.30 | ±1.60 | 0.16 | ±0.06 |
| 7086.28 | ±1.01 | 0.21 | ±0.02 | 8492.05 | ±1.32 | 0.21 | ±0.11 | 11055.36 | ±2.79 | 0.04 | ±0.01 |
| | | | | 8895.01 | ±1.55 | 0.59 | ±0.30 | 11898.19 | ±2.17 | 0.15 | ±0.03 |

As described above, optimal embodiments of the present invention have been disclosed in the drawings and the specification. Although specific terms have been used in the present specification, these are merely intended to describe the present invention and are not intended to limit the meanings thereof or the scope of the present invention described in the accompanying claims. Therefore, those skilled in the art will appreciate that various modifications and other equivalent embodiments are possible from the embodiments. Therefore, the technical scope of the present invention should be defined by the technical spirit of the claims.

What is claimed is:

1. A method of identifying a Bacillus species using a matrix-assisted laser desorption/ionization time-of-flight mass spectrometer (MALDI-TOF MS), comprising:
directly spotting a sample containing an intact spore-forming Bacillus bacterium onto a MALDI target plate without conducting any pretreatment thereto; and
performing MALDI-TOF mass spectroscopy to acquire spectral data of the sample; and
analyzing the spectral data with reference to a reference data (m/z) to identify the sample, said reference data being established for biomarker peaks distinctive for known Bacillus species.

2. The method of claim 1, further comprising applying a matrix solution to the sample and drying it after the spotting step.

3. The method of claim 2, wherein the matrix solution comprise a matrix material selected from the group consisting of dihydroxybenzoic acid, sinapinic acid, trihydroxy acetophenone, hydroxyphenylazo benzoic acid, dithranol, cyano-hydroxycinnamic acid, all-trans-retinoic acid, indoleacrylic acid, and a combination thereof.

4. The method of claim 3, wherein the matrix material is cyano-hydroxycinnamic acid.

5. The method of claim 1, wherein the MALDI-TOF MS is an Autoflex Speed LRF mass spectrometer from Brucker.

6. The method of claim 5, wherein the MALDI-TOF MS uses an MBT_FC parameter with a laser power of 50% or higher.

7. The method of claim 5, wherein the MALDI-TOF MS uses an MBT^{—}FC parameter with a laser power at least 1.6-fold larger than that necessary for analyzing E. coli.

8. The method of claim 6, wherein the MALDI-TOF MS uses an MBT_FC parameter with a laser power of from 70% to 80%.

9. The method of claim 7, wherein the MALDI-TOF MS uses an MBT_FC parameter with a laser power 2.5- to 2.7-fold larger than that necessary for analyzing E. coli.

10. The method of claim 1, in the Bacillus species is selected from the group consisting of Bacillus anthracis, Bacillus cereus, Bacillus globigii, Bacillus subtilis, Bacillus thuringiensis, and a combination thereof.

11. The method of claim 1, wherein the spectral data is obtained in consideration of a standard error after 20 rounds of MALDI-TOF mass spectrometry.

12. The method of claim 11, wherein the reference data (m/z) established for biomarker peaks distinctive for known Bacillus species is at least one selected from the group consisting of:
(1) 2196, 2473, 2503, 2786, 3089, 3376, 3594, 6684, 6753, 6840, and 9746;
(2) 3357, 3419, 3709, 4836, 4953, 6714, 6839, and 7085;
(3) 2324, 2870, 2886, 2992, 3123, 4447, 7907, 8053, 8199, 8345, 8492, and 8895;
(4) 2324, 2720, 2871, 2887, 2935, 3116, 5299, 5948, 7338, 8201, 8347, 8494, 8896, and 11898; and
(5) 2109, 2127, 3357, 3419, 3709, 6715, 6840, and 7086

13. The method of claim 11, wherein the reference data (m/z) established for biomarker peaks distinctive for known Bacillus species is at least one selected from the group consisting of:
(1) 2080, 2097, 2196, 2446, 2473, 2503, 2518, 2523, 2579, 2786 3075, 3089, 3150, 3341, 3376, 3576, 3594, 3653, 4031, 4196, 4328, 4383, 4554, 4956, 5263, 5541, 6684, 6699, 6753, 6840 and 9746;
(2) 2109, 2123, 3079, 3193, 3357, 3419, 3542, 3709, 3807, 4031, 4335, 4425, 4836, 4953, 5173, 6714, 6839 and 7085;
(3) 2324, 2870, 2886, 2918, 2934, 2992, 3123, 3430, 3760, 4418, 4447, 4682, 5047, 7072, 7336, 7907, 8053, 8199, 8345, 8492 and 8895;
(4) 2324, 2720, 2871, 2887, 2919, 2935, 2991, 3116, 3528, 4448, 5048, 5299, 5948, 7073, 7338, 8201, 8347, 8494, 8896, 11056 and 11898; and
(5) 2109, 2127, 2266, 2380, 2529, 3095, 3357, 3419, 3542, 3709, 4031, 4335, 4425, 4837, 4971, 5173, 6352, 6715, 6840 and 7086.

14. The method of claim 12, wherein the sample is identified as Bacillus anthracis when the peaks are detected at the mass-to-charge ratios (m/z) of group (1).

15. The method of claim 12, wherein the sample is identified as Bacillus cereus when the peaks are detected at the mass-to-charge ratios (m/z) of group (2).

16. The method claim 12, wherein the sample is identified as Bacillus globigii when the peaks are detected at the mass-to-charge ratios (m/z) of group (3).

17. The method of claim 12, wherein the sample is identified as Bacillus subtilis when the peaks are detected at the mass-to-charge ratios (m/z) of group (4).

18. The method of claim 12, wherein the sample is identified as Bacillus thuringiensis when the peaks are detected at the mass-to-charge ratios (m/z) of group (5).
